(19) United States
(12) Patent Application Publication
(10) Pub. No.: US 2010/0069681 A1
(43) Pub. Date: Mar. 18, 2010

(54) PROCESS FOR PREPARING 3-ALKOXYPROPAN-1-OLS

(75) Inventors: Jens Heimann, Worms (DE); Markus Roesch, Oppenheim (DE); Ellen Dahlhoff, Limburgerhof (DE); Steffen Maas, Bubenheim (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, LUDWIGSHAFEN (DE)

(21) Appl. No.: 12/516,211

(22) PCT Filed: Nov. 12, 2007
(86) PCT No.: PCT/EP07/62194

§ 371 Date: May 26, 2009

(30) Foreign Application Priority Data
Nov. 27, 2006 (EP) 06124819.1

(51) Int. Cl. C07C 41/00

(52) U.S. Cl. 568/678

(57) ABSTRACT

The present invention relates to a process for preparing 3-alkoxypropan-1-ols of the general formula I

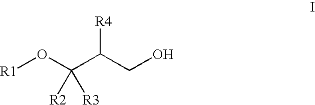

by catalytic hydrogenation of esters of the general formula II,

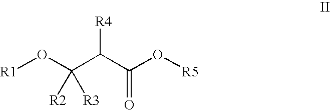

where the radicals R1 and R2 and R3, R4 and R5 are each, independently of one another, a straight-chain or branched C₁-C₂₀-alkyl radical which may optionally be substituted by one or more C₁-C₂₀-alkoxy radicals or interrupted by one or more oxygen atoms in the chain, C₆-C₂₀-aryl, C₇-C₂₀-arylalkyl which may optionally be substituted by one or more C₁-C₂₀-alkoxy radicals or interrupted by one or more oxygen atoms in the alkyl chain, C₇-C₂₀-alkylaryl which may optionally be substituted by one or more C₁-C₂₀-alkoxy radicals or interrupted by one or more oxygen atoms in the alkyl chain, a C₁-C₂₀-cycloalkyl radical which may optionally be substituted by one or more C₁-C₂₀-alkoxy radicals and R2, R3 and R4 can, independently of one another, also be hydrogen, over chromium- and nickel-free catalysts.

The present invention relates to a process for preparing 3-alkoxypropan-1-ols of the general formula I

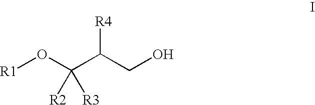

by catalytic hydrogenation of esters of the general formula II,

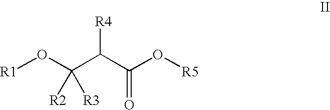

where the radicals R1 and R2 and R3, R4 and R5 are each, independently of one another, a straight-chain or branched C₁-C₂₀-alkyl radical which may optionally be substituted by one or more C₁-C₂₀-alkoxy radicals or interrupted by one or more oxygen atoms in the chain, C₆-C₂₀-aryl, C₇-C₂₀-arylalkyl which may optionally be substituted by one or more C₁-C₂₀-alkoxy radicals or interrupted by one or more oxygen atoms in the alkyl chain, C₇-C₂₀-alkylaryl which may optionally be substituted by one or more C₁-C₂₀-alkoxy radicals or interrupted by one or more oxygen atoms in the alkyl chain, a C₇-C₂₀-cycloalkyl radical which may optionally be substituted by one or more C₁-C₂₀-alkoxy radicals and R2, R3 and R4 can, independently of one another, also be hydrogen, over chromium- and nickel-free catalysts.

3-Alkoxypropanols are sought-after solvents and additionally have a variety of uses as building blocks for active compounds.

Various methods, which generally start out from a C₃ building block, are known for preparing 3-alkoxypropanols.

JP-A-2001247503 discloses the ZrO₂-catalyzed addition of alcohols onto allyl alcohol, but this proceeds with only an unsatisfactory conversion and unsatisfactory selectivity.

The etherification of the corresponding diols described in JP-A-2004196783 either requires stoichiometric amounts of base or conversion and selectivity to the monoether are not satisfactory.

It is also known that acrolein can firstly be alkoxylated in the presence of a catalyst to form 3-alkoxypropionaldehyde and the aldehyde can then be hydrogenated in the presence of hydrogenation catalysts to give the 3-alkoxypropanol. Nickel catalysts are recommended as particularly effective and inexpensive catalysts for this hydrogenation, for example in EP-A 1 085 003. These processes have the disadvantage that the catalytically active element, particularly when using Raney nickel catalysts, contaminates the product stream in small amounts in the form of soluble compounds and additional work-up steps are therefore required. There are additional concerns about the use of nickel catalysts and acrolein because of their toxicity. Particular safety precautions are necessary for production on an industrial scale.

Although the catalytic hydrogenation of optionally substituted 1,3-dioxane described, for example, in EP-A 810 194 makes do without the use of nickel catalysts, the conversions and selectivities which can be achieved are not satisfactory.

Furthermore, Mozingo and Folkers describe the hydrogenation of ethyl 3-ethoxypropionate in methanol over a copper chromite catalyst at 290-438 bar to form 3-ethoxypropanol and ethanol in J. Am. Chem. Soc. 1948, 70, 227-229. Both the high reaction pressure which incurs high capital costs and also the use of a chromium-comprising catalyst which is known to be toxic are disadvantages.

Proceeding from this prior art, it was an object of the present invention to provide a process by means of which 3-alkoxypropan-1-ols can be prepared with good conversion and good selectivity without the use of toxicologically problematical substances and can be realized in a technically simple manner and is generally applicable, i.e. can also be carried out in industrial multipurpose plants, without occurrence of the abovementioned disadvantages.

We have now found a process for preparing 3-alkoxypropan-1-ols of the general formula I

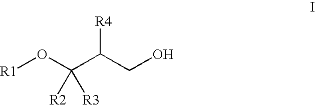

by catalytic hydrogenation of esters of the general formula II,

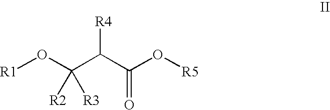

where the radicals R1 and R2 and R3, R4 and R5 are each, independently of one another, a straight-chain or branched C₁-C₂₀-alkyl radical which may optionally be substituted by one or more C₁-C₂₀-alkoxy radicals or interrupted by one or more oxygen atoms in the chain, C₆-C₂₀-aryl, C₇-C₂₀-arylalkyl which may optionally be substituted by one or more C₁-C₂₀-alkoxy radicals or interrupted by one or more oxygen atoms in the alkyl chain, C₇-C₂₀-alkylaryl which may optionally be substituted by one or more C₁-C₂₀-alkoxy radicals or interrupted by one or more oxygen atoms in the alkyl chain, a C₇-C₂₀-cycloalkyl radical which may optionally be substituted by one or more C₁-C₂₀-alkoxy radicals and R2, R3 and R4 can, independently of one another, also be hydrogen, over chromium- and nickel-free catalysts.

The process of the invention allows the selective conversion of the corresponding esters of the general formula II, which can be obtained by addition of alcohols onto acrylic esters, into 3-alkoxypropan-1-ols with a high degree of conversion. The process can also be carried out in industrial plants which are utilized for preparing changing products without particular safety precautions.

Catalysts used according to the invention for the hydrogenation can be homogeneous or heterogeneous metal-comprising catalysts in which the metal is present in elemental form or in the form of a compound; chromium and nickel are not comprised.

The catalysts which can be used preferably comprise at least one metal of group 7, 8, 9, 10, 11 or 14 of the Periodic Table of the Elements. The catalysts which can be used according to the invention more preferably comprise at least one element selected from the group consisting of Re, Fe, Ru, Co, Rh, Ir, Pd, Pt, Cu and Au. The catalysts which can be used according to the invention particularly preferably comprise at least one element selected from the group consisting of Pd, Pt, Ru and Cu. The catalysts which can be used according to the invention most preferably comprise Cu as hydrogenation-active component.

Particularly preferred catalysts comprising Cu as hydrogenation-active component can be produced as disclosed in U.S. Pat. No. 5,403,962 or WO 2004/85356, which are hereby expressly incorporated by reference.

Particular preference is therefore given to using the shaped catalyst bodies which are known from WO 2004/85356 and can be produced by a process in which
(i) an oxidic material comprising copper oxide, aluminum oxide and at least one of the oxides of lanthanum, tungsten, molybdenum, titanium or zirconium is provided,
(ii) pulverulent metallic copper, copper flakes, pulverulent cement or graphite or a mixture thereof can be added to the oxidic material and
(iii) the mixture resulting from (ii) is shaped to give a shaped body.

Among the oxides of lanthanum, tungsten, molybdenum, titanium or zirconium, lanthanum oxide is preferred.

Very particular preference is given to catalysts as disclosed in WO 2004/85356, which is hereby expressly incorporated by reference, in which the oxidic material comprises
(a) copper oxide in a proportion in the range 50≦x≦80% by weight, preferably 55≦x≦75% by weight,
(b) aluminum oxide in a proportion in the range 15≦y≦35% by weight, preferably 20≦y≦30% by weight, and
(c) at least one of the oxides of lanthanum, tungsten, molybdenum, titanium or zirconium in a proportion in the range 2≦z≦20% by weight, preferably 3≦z≦15% by weight,
   in each case based on the total weight of the oxidic material after calcination, where 80≦x+y+z≦100, in particular 95≦x+y+z≦100.

Homogeneous catalysts comprising at least one element of group 8, 9 or 10 with the exception of nickel are also suitable. Further preference is given to homogeneous catalysts comprising Ru, Rh, and/or Ir.

If compounds of the abovementioned elements are used, suitable compounds are, for example, salts such as halides, oxides, nitrates, sulfates, carbonates, alkoxides and aryloxides, carboxylates, acetylacetonates or acetates of the respective metal. Furthermore, these salts can be modified with complexing ligands. The catalysts utilized according to the invention preferably comprise one or more oxygen-, sulfur-, nitrogen- or phosphorus-comprising complexing ligands.

The catalyst is preferably selected from among halides, oxides, nitrates, sulfates, carbonates, alkoxides, aryloxides, carboxylates, acetylacetonates and acetates of the respective metal and also the compounds of the metal comprising CO, CS, an optionally organyl-substituted amino ligand, an optionally organyl-substituted phosphine ligand, an alkyl, allyl, cyclopentadienyl and/or olefin ligand.

Mention may here be made by way of example of, for instance, RhCl(TPP)₃ or Ru₄H₄(CO)₁₂. Particular preference is given to homogeneous catalysts comprising Ru. For example, use is made of homogeneous catalysts as are described in U.S. Pat. No. 5,180,870, U.S. Pat. No. 5,321,176, U.S. Pat. No. 5,177,278, U.S. Pat. No. 3,804,914, U.S. Pat. No. 5,210,349, U.S. Pat. No. 5,128,296 and in D. R. Fahey in J. Org. Chem. 38 (1973) p. 80-87, whose relevant disclosure is fully incorporated by reference into the present patent application. Such catalysts are, for instance, (TPP)₂(CO)₃Ru, [Ru(CO)₄]₃, (TPP)₂Ru(CO)₂Cl₂, (TPP)₃(CO)RuH₂, (TPP)₂(CO)₂RuH₂, (TPP)₂(CO)₂RuClH or (TPP)₃(CO)RuCl₂.

It is particularly useful to employ at least one heterogeneous catalyst which can comprise at least one of the abovementioned metals as such, as Raney catalyst and/or applied to a customary support. Preferred support materials are, for instance, activated carbons or oxides such as aluminum oxides, silicon oxides, titanium oxides or zirconium oxides. Mention may likewise be made of, inter alia, bentonites as support materials. If two or more metals are used, they can be present either separately or as an alloy. It is possible to use at least one metal as such and at least one other metal as Raney catalyst or at least one metal as such and at least one other metal applied to at least one support, or at least one metal as Raney catalyst and at least one other metal applied to at least one support, or at least one metal as such and at least one other metal as Raney catalyst and at least one other metal applied to at least one support.

The catalysts used can, for example, also be precipitated catalysts. Such catalysts can be produced by precipitating their catalytically active components from their salt solutions, in particular from the solutions of their nitrates and/or acetates, for example by addition of solutions of alkali metal and/or alkaline earth metal hydroxide and/or carbonate solutions, for example as sparingly soluble hydroxides, oxide hydrates, basic salts or carbonates, subsequently drying the precipitates obtained and then converting these into the corresponding oxides, mixed oxides and/or mixed-valence oxides by calcination at generally from 300 to 700° C., in particular from 400 to 600° C., and reducing the oxides to the corresponding metals and/or oxidic compounds having a lower oxidation state by treatment with hydrogen or with hydrogen-comprising gases at generally from 50 to 700° C., in particular from 100 to 400° C., so as to produce the actual catalytically active form. The reduction is generally continued until no more water is formed. In the production of precipitated catalysts which comprise a support material, the precipitation of the catalytically active components can be carried out in the presence of the support material concerned. The catalytically active components can advantageously be precipitated simultaneously with the support material from the appropriate salt solutions.

Preference is given to using hydrogenation catalysts which comprise the metals or metal compounds which catalyze the hydrogenation deposited on a support material.

Apart from the abovementioned precipitated catalysts which additionally comprise a support material in addition to the catalytically active components, support materials in which the catalytic hydrogenation-active component has been applied, for example by impregnation, to a support material are generally also suitable for the process of the invention.

The way in which the catalytically active metal is applied to the support is generally not critical and the application can be achieved in various ways. The catalytically active metals can, for example, be applied to these support materials by impregnation with solutions or suspensions of the salts or oxides of the elements concerned, drying and subsequent reduction of the metal compounds to the corresponding metals or compounds having a lower oxidation state by means of a reducing agent, preferably by means of hydrogen or complex hydrides. Another possible way of applying the catalytically active metals to these supports is to impregnate the supports with solutions of salts of the catalytically active metals which can easily be decomposed thermally, for example nitrates, or complexes of the catalytically active metals which can easily be decomposed thermally, for example carbonyl or hydrido complexes, and to heat the support which has been impregnated in this way to temperatures in the range from 300 to 600° C. to thermally decompose the adsorbed metal compounds. This thermal decomposition is preferably carried out under a protective gas atmosphere. Suitable protective gases are, for example, nitrogen, carbon dioxide, hydrogen or the noble gases. Furthermore, the catalytically active metals can be deposited on the catalyst support by vapor deposition or by flame spraying. The content of catalytically active metals in these supported catalysts is in principle not critical for the success of the process of the invention. In general, higher contents of catalytically active metals in these supported catalysts lead to higher space-time yields than do lower contents.

In general, supported catalysts whose content of catalytically active metals is in the range from 0.1 to 90% by weight, preferably in the range from 0.5 to 80% by weight, based on the total weight of the catalyst, are used. Since these indicated contents are based on the total catalyst including support material but the various support materials have very different specific gravities and specific surface areas, it is also conceivable for the contents to be below or above the figures given without this having an adverse effect on the result obtained in the process of the invention. Of course, it is also possible for a plurality of the catalytically active metals to be applied to the respective support material. Furthermore, the catalytically active metals can be applied to the support by, for example, the method of DE-OS 25 19 817 or EP 0 285 420 A1. In the catalysts described in the abovementioned documents, the catalytically active metals are present as alloys which are produced by thermal treatment and/or reduction of, for example, the support material which has been impregnated with a salt or complex of the abovementioned metals.

Both the activation of the precipitated catalysts and also that of the supported catalysts can also be effected in situ at the beginning of the reaction by the hydrogen present. These catalysts are preferably activated separately before use. As support materials, it is generally possible to use the oxides of zinc, aluminum and titanium, zirconium dioxide, silicon dioxide, lanthanum oxide, clays such as montmorillonites, silicates such as magnesium or aluminum silicates, zeolites such as the structure types ZSM-5 or ZSM-10 or activated carbon. Preferred support materials are aluminum oxides, titanium dioxides, silicon dioxide, zirconium dioxide, lanthanum oxide and activated carbon. Of course, mixtures of various support materials can also serve as supports for catalysts which can be used in the process of the invention. As additives for the targeted setting of the acidic or in particular basic properties of the catalyst, compounds comprising alkali and/or alkaline earth metals, preferably oxides, can also be comprised. Possible catalysts also include ones comprising zinc oxide or zirconium oxide as active component.

In the process of the invention, the heterogeneous catalyst can, for example, be used as suspended catalyst and/or as fixed-bed catalyst.

If, for example, the hydrogenation in the process of the invention is carried out using at least one suspended catalyst, the hydrogenation is preferably carried out in at least one stirred reactor or in at least one bubble column or in at least one packed bubble column or in a combination of two or more identical or different reactors.

The term “different reactors” refers in the present context both to different types of reactor and to reactors of the same type which differ, for example, in terms of their geometry, for example their volume and/or their cross section, and/or the hydrogenation conditions in the reactors.

If, for example, a heterogeneous catalyst is used as suspended catalyst in the hydrogenation, this is, for the purposes of the present invention, preferably separated off by means of at least one filtration step. The catalyst which has been separated off in this way can be recirculated to the hydrogenation or be passed to at least one other process of any desired type. It is likewise possible to work up the catalyst, for example in order to recover the metal comprised in the catalyst.

In the process of the invention, the hydrogenation is particularly preferably carried out using at least one fixed-bed catalyst. For this purpose, preference is given to using at least one tube reactor such as at least one shaft reactor and/or at least one shell-and-tube reactor, with any individual reactor being able to be operated in the upflow or downflow mode. When two or more reactors are used, at least one can be operated in the upflow mode and at least one can be operated in the downflow mode.

In one embodiment, the solution to be hydrogenated is pumped in a single pass over the catalyst bed. In another embodiment of the process of the invention, part of the product obtained after passage through the reactor is continuously taken off as product stream and, if appropriate, passed through a second reactor. The other part of the product (recycle stream) is fed back into the reactor together with fresh starting material (feed stream). This liquid circuit serves, inter alia, to remove heat. This mode of operation is also referred to as recycle mode. In the process of the invention, preference is given to setting a weight ratio of feed to recycle stream of from 3:1 to 1:40, particularly preferably from 2:1 to 1:10.

Apart from the liquid recycle stream, part of the gas phase of the output after phase separation can optionally also be recirculated, i.e. be mixed with the feed and the fresh hydrogen gas at any point upstream of the reactor inlet. This can be achieved either after separating off the liquid phase of the output by means of a second separate pump or together with the liquid recycle by means of one pump. Particular preference is given to recirculation of the gas phase (recycle gas) for removal of heat in the case of the particularly preferred small liquid recycle ratios of feed:recycle stream=3:1 to 1:10.

Before use in any process, for example before recirculation to the process of the invention, both the at least one homogeneous catalyst and the at least one heterogeneous catalyst can be regenerated by at least one suitable method, should this be necessary.

Preference is given to heterogeneous catalysts which are present in shaped bodies in the form of pellets, rings, cylinders, spheres, trilobes or extrudates for the hydrogenation.

Heterogeneous catalysts are, if necessary, generally activated, preferably by means of hydrogen, before use. The methods of achieving this are known to those skilled in the art.

The catalytic hydrogenation according to the invention is carried out at from 10 to 400 bar, preferably from 120 to 280 bar and particularly preferably from 130 to 250 bar, and a temperature of from 100 to 300° C., preferably from 120 to 200° C. and particularly preferably from 130 to 190° C.

The ester II which serves as starting material for the hydrogenation according to the invention can be prepared by processes known per se. Thus, for example, the ester II can be prepared by addition of alcohols of the formula R1-OH onto acrylic esters of the formula III,

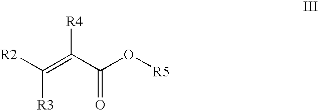

where the radicals R1 and R2 and R3, R4 and R5 are each, independently of one another, a straight-chain or branched C₁-C₂₀-alkyl radical which may optionally be substituted by one or more C₁-C₂₀-alkoxy radicals or interrupted by one or more oxygen atoms in the chain, C₆-C₂₀-aryl, C₇-C₂₀-arylalkyl which may optionally be substituted by one or more C₁-C₂₀-alkoxy radicals or interrupted by one or more oxygen atoms in the alkyl chain, C₇-C₂₀-alkylaryl which may optionally be substituted by one or more C₁-C₂₀-alkoxy radicals or interrupted by one or more oxygen atoms in the alkyl chain, a C₇-C₂₀-cycloalkyl radical which may optionally be substituted by one or more C₁-C₂₀-alkoxy radicals and R2, R3 and R4 can, independently of one another, also be hydrogen.

The addition of the alcohol onto the acrylic ester can be carried out in an inert solvent such as tetrahydrofuran or ethylene glycol dimethyl ether. This is necessary particularly when the alcohol R1-OH used or the acrylic ester III is not liquid under the conditions of the addition reactions.

In a preferred variant of the preparation of the starting material II for the hydrogenation according to the invention, from 0.001 to 10 mol %, based on the amount of acrylic ester, of metal alkoxides which are soluble in the reaction mixture, e.g. sodium methoxide, are used as catalyst in the addition reaction. In a particularly preferred embodiment, the anion of the metal alkoxide corresponds to the anion of the alcohol R1-OH to be added on.

In a further preferred embodiment, the acrylic ester III and the alcohol R1-OH are selected so that R5 and R1 are the same radical. In this way, a reduction in the yield of II caused by transesterification and consequently liberation of alcohol R5-OH can be avoided.

In a preferred variant of the invention, the ester II is not purified by methods known per se for the purification of substances, e.g. distillation, but instead the reaction mixture from the addition reaction is, after removal or neutralization of the addition catalyst, used without further purification of the ester II directly in the hydrogenation.

If homogeneous basic or acidic catalysts have been used for the preparation of the ester II, these are preferably neutralized with an organic or inorganic acid, for example formic acid, acetic acid or another monocarboxylic or dicarboxylic acid, or an organic or inorganic base and/or, if appropriate, filtered off. Furthermore, the catalyst used for the addition reaction can also be removed by means of a suitable ion exchanger, in particular a commercial anion- or cation-exchange resin.

If a heterogeneous catalyst has been used, the reaction mixture has to be filtered before being used as starter material in the hydrogenation according to the invention.

Apart from the alkoxypropan-1-ol l, n-propanol in particular is formed as by-product in the hydrogenation process of the invention by hydrogenation of acrylic ester formed by redissociation; this n-propanol can easily be separated off by distillation and either used further or utilized in another way.

The invention is illustrated by the following examples but is not restricted thereby.

### EXAMPLES

### Example 1

### Preparation of 6-ethyl-4-oxadecan-1-ol

1670 g of 2-ethylhexanol were reacted with 640 mg of sodium at 60° C. After a homogeneous solution had been formed, 1160 g of 2-ethylhexyl acrylate were introduced over a period of 6 hours. After a further 6 hours at 60° C., the reaction mixture was passed through an acid cation exchanger (Amberlite IR-120 from Merck, Darmstadt) which had been freshly regenerated with 2N sulfuric acid and washed with methanol and then with 2-ethylhexanol. A 67% strength by weight solution of 2-ethylhexyl 6-ethyl-4-oxadecanoate in 2-ethylhexanol was obtained (yield 96%).

80 g of copper catalyst having the composition 57% of CuO/28.5% of Al₂O₃/9.5% of La₂O₃/5% of Cu as 3×3 mm pellets produced as described in Example 1 of WO-A 2004/85356 were reduced in a stream of hydrogen at 180° C. Hydrogenation was subsequently carried out at 180° C., 200 bar, a feed rate of 12 g/h of the reaction mixture comprising 2-ethylhexyl 6-ethyl-4-oxadecanoate prepared above, recirculation of the output to the reactor inlet in an amount of 80 g/h and a hydrogen feed rate of 40 standard l/h. The output comprised, at a conversion of 99%: 68% by weight of 2-ethylhexanol, 24% by weight of 6-ethyl-4-oxadecan-1-ol (yield 59%), 5% by weight of propanol, 1% by weight of 2-ethylhexyl 6-ethyl-4-oxadecanoate and a total of 2% by weight of other compounds.

### Example 2

### Preparation of 3-methoxypropan-1-ol

1160 g of methyl acrylate were added over a period of 6 hours to 1160 g of methanol and 5 g of a 30% strength solution of sodium methoxide in methanol at 60° C. and the mixture was subsequently stirred at 60° C. for a further two hours. A methyl acrylate conversion of more than 99%, corresponding to a yield of 99%, was determined by gas chromatography. The mixture was neutralized at room temperature with one equivalent of acetic acid based on the amount of sodium methoxide and the reaction solution was used directly as feed for the continuous hydrogenation.

80 g of copper catalyst having the composition 57% of CuO/28.5% of Al₂O₃/9.5% of La₂O₃/5% of Cu as 3×3 mm pellets produced as described in Example 1 of WO-A 2004/85356 were reduced in a stream of hydrogen at 180° C. The plant was subsequently operated under the following conditions: temperature in the reactor 170° C., pressure 200 bar, feed rate of 16 g/h of the ester-comprising reaction mixture prepared above, recirculation of the output to the reactor inlet in an amount of 80 g/h and a hydrogen feed rate of 40 standard l/h. The output comprised, at a conversion of 99%: 57% by weight of methanol, 27% by weight of 3-methoxypropan-1-ol, corresponding to a yield of 51%, 14% by weight of propanol, 1% by weight of methyl 3-methoxypropionate and a total of 1% by weight of other esters.

1. A process for preparing 3-alkoxypropan-1-ols of formula I 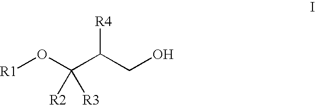
by catalytic hydrogenation of esters of formula II, 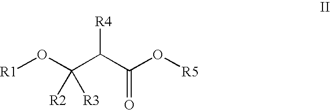
in the presence of at least one chromium-free and nickel-free catalyst,
where each of R1, R2, R3, R4 and R5 is, independently, a straight-chain or branched C₁-C₂₀-alkyl radical which may optionally be substituted by one or more C₁-C₂₀-alkoxy radicals or interrupted by one or more oxygen atoms in the chain, C₆-C₂₀-aryl, C₇-C₂₀-arylalkyl which may optionally be substituted by one or more C₁-C₂₀-alkoxy radicals or interrupted by one or more oxygen atoms in the alkyl chain, C₇-C₂₀-alkylaryl which may optionally be substituted by one or more C₁-C₂₀-alkoxy radicals or interrupted by one or more oxygen atoms in the alkyl chain, a C₇-C₂₀-cycloalkyl radical which may optionally be substituted by one or more C₁-C₂₀-alkoxy radicals and each of R2, R3 and R4 can, independently, also be hydrogen.

2. The process according to claim 1, wherein the at least one chromium-free and nickel-free catalyst is a homogeneous catalyst which is a salt, a compound comprising one or more oxygen-, sulfur-, nitrogen- or phosphorus-comprising complexing ligands of a metal of groups 8, 9 and 10 of the Periodic Table with the exception of nickel, or a combination thereof.

3. The process according to claim 1, wherein the at least one chromium-free and nickel-free catalyst is a heterogeneous catalyst comprising at least one metal selected from groups 7, 8, 9, 10 with the exception of nickel, 11 and 14 of the Periodic Table of the Elements.

4. The process according to claim 3, wherein the at least one chromium-free and nickel-free catalyst is a shaped body which can be produced by
obtaining an oxidic material comprising copper oxide, aluminum oxide and at least one of the oxides of lanthanum, tungsten, molybdenum, titanium or zirconium,
(ii) adding pulverulent metallic copper, copper flakes, pulverulent cement, graphite or a mixture thereof to the oxidic material and
(iii) shaping the mixture resulting from (ii) to give a shaped body.

5. The process according to claim 4, wherein the oxidic material comprises
(a) copper oxide in a proportion in the range 50≦x≦80% by weight,
(b) aluminum oxide in a proportion in the range 15≦y≦35% by weight, and
(c) at least one of the oxides of lanthanum, tungsten, molybdenum, titanium or zirconium in a proportion in the range 2≦z≦20% by weight,
in each case based on the total weight of the oxidic material after calcination, where 80≦x+y+z≦100 with cement not included in the oxidic material in the above sense.

6. The process according to claim 1, wherein the hydrogenation is carried out in the liquid phase at a pressure of from 100 to 400 bar and a temperature of from 100 to 300° C.

7. The process according to claim 1, wherein the process is operated in the recycle mode at a ratio of recycle:feed of from 1:3 to 40:1.

8. The process according to claim 1, wherein a reaction mixture which comprises the ester of formula II and has been prepared by addition of alcohols of formula R1-OH onto acrylic esters of formula III, 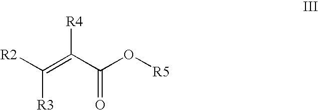
is present as a starting material,
where each of R1, R2, R3, R4 and R5 is, independently, a straight-chain or branched C₁-C₂₀-alkyl radical which may optionally be substituted by one or more C₁-C₂₀-alkoxy radicals or interrupted by one or more oxygen atoms in the chain, C₆-₂₀-aryl, C₇-C₂₀-arylalkyl which may optionally be substituted by one or more C₁-C₂₀-alkoxy radicals or interrupted by one or more oxygen atoms in the alkyl chain, C₇-C₂₀-alkylaryl which may optionally be substituted by one or more C₁-C₂₀-alkoxy radicals or interrupted by one or more oxygen atoms in the alkyl chain, a C₇-C₂₀-cycloalkyl radical which may optionally be substituted by one or more C₁-C₂₀-alkoxy radicals and each of R2, R3 and R4 can, independently, also be hydrogen.

9. The process according to claim 8, wherein the addition of the alcohols R1-OH onto acrylic esters of formula III is carried out in the presence of catalytic amounts of a metal alkoxide.

10. The process according to claim 8, wherein the anion of the metal alkoxide corresponds to the anion of the alcohol R1-OH to be added on.

11. The process according to claim 8, wherein an acrylic ester of formula III and an alcohol of the formula R1-OH in which the radicals R5 and R1 are identical are present.

12. The process according to claim 8, wherein the reaction mixture comprising the ester II is not purified before hydrogenation.

13. The process according to claim 8, wherein the reaction mixture comprising the ester II is treated with a cation exchanger or neutralized with an organic or inorganic acid before hydrogenation.

14. The process according to claim 3, wherein the at least one chromium-free and nickel-free catalyst is a shaped body which can be produced by
(i) adding pulverulent metallic copper, copper flakes, pulverulent cement, graphite or a mixture thereof to an oxidic material comprising copper oxide, aluminum oxide and at least one of the oxides of lanthanum, tungsten, molybdenum, titanium or zirconium and
(ii) shaping the mixture resulting from (i) to give a shaped body.

15. The process according to claim 4, wherein the oxidic material comprises
(a) copper oxide in a proportion in the range 55≦x≦75% by weight,
(b) aluminum oxide in a proportion in the range 20≦y≦30% by weight, and
(c) at least one of the oxides of lanthanum, tungsten, molybdenum, titanium or zirconium in a proportion in the range 3≦z≦15% by weight,
in each case based on the total weight of the oxidic material after calcination, where 95≦x+y+z≦100 with cement not being included in the oxidic material in the above sense.
